(19) United States
(12) Plant Patent Application Publication
(10) Pub. No.: US 2026/0240059 P1
(43) Pub. Date: Aug. 13, 2026

(54) Fragaria Plant Named 'Florencia'

(50) ;

(71) Applicant: Flevo Berry Holding B.V., Ens (NL)

(72) Inventor: Marcel Suiker, Ens (NL)

(73) Assignee: Flevo Berry Holding B.V., Ens (NL)

(21) Appl. No.: 19/395,677

(22) Filed: Nov. 20, 2025

(30) Foreign Application Priority Data
Nov. 20, 2024 (QZ) 2024/2688

(51) Int. Cl. A01H 6/74; A01H 5/08

(52) U.S. Cl. CPC A01H 6/7409; A01H 5/08
USPC PLT/208

(57) ABSTRACT

A new and distinct Fragaria x ananassa plant named ‘Florencia’ which exhibits an upright and densely foliaged growth habit with large trifoliate leaves, partially remontant fruiting with an abundance of large, long-conical red berries produced from late spring through autumn, berries with a taste that is best described as a pleasant blend of slightly sweet and moderately acidic flavors, and good resistance to powdery mildew. ‘Florencia’ has shown the stability of these characteristics from generation to generation.

Latin name of the genus and species:

The Latin name of the genus and species of the novel variety disclosed herein is Fragaria x ananassa Duchesne ex Rozier.

Variety denomination:

The inventive cultivar of Fragaria plant disclosed herein has been given the variety denomination ‘Florencia’.

### BACKGROUND OF THE INVENTION

Parentage: ‘Florencia’ is a seedling selection that resulted from a controlled pollination breeding program carried out in 2018 in Ens, Netherlands. The new variety originated as a seedling from the crossing of Fragaria x ananassa ‘14-168’ (not patented), the seed parent, with Fragaria x ananassa ‘14-149’ (not patented) as the pollen parent. Seed from said cross was harvested, then germinated, and the resulting seedlings were grown to a mature size in order to evaluate for desirable commercial characteristics. The claimed plant was then subjected to a series of trials from 2018 through 2023 to evaluate the distinctness and stability of the characteristics first observed. In 2024, the claimed plant was finally selected as a new and distinctive cultivar.

Asexual Reproduction: Asexual reproduction of ‘Florencia’, by way of rooting stolon cuttings, was first performed in September of 2018 in Ens, Netherlands. Through seven subsequent generations, the unique features of this cultivar have proven to be stable and true to type.

### SUMMARY OF THE INVENTION

The cultivar ‘Florencia’ has not been observed under all possible environmental conditions, thus being so the phenotype may vary somewhat with variations in environment such as temperature, light intensity, and water availability, with no variance in genotype. The following traits have been repeatedly observed and are determined to be the unique characteristics of ‘Florencia’. These characteristics in combination distinguish ‘Florencia’ as a new and distinct Fragaria cultivar:
1. Fragaria ‘Florencia’ exhibits an upright, densely foliaged growth habit with a relative abundance of stolons bearing large trifoliate leaves; and
2. Fragaria ‘Florencia’ exhibits partially remontant fruiting with an abundance of white flowers that bear fruit from late spring through autumn; and
3. Fragaria ‘Florencia’ exhibits large, long conical, vibrant red berries with level to slightly recessed achenes; and
4. Fragaria ‘Florencia’ exhibits a taste that is best described as a pleasant blend of slightly sweet and moderately acidic flavors; and
5. Fragaria ‘Florencia’ exhibits good resistance to powdery mildew.

### BRIEF DESCRIPTION OF THE FIGURES

FIG. 1 illustrates, as nearly true as it is reasonably possible to make the same in color photographs of this type, an exemplary 12-month-old ‘Florencia’ plant, grown at a commercial greenhouse in Ens, Netherlands in a 20 cm container.

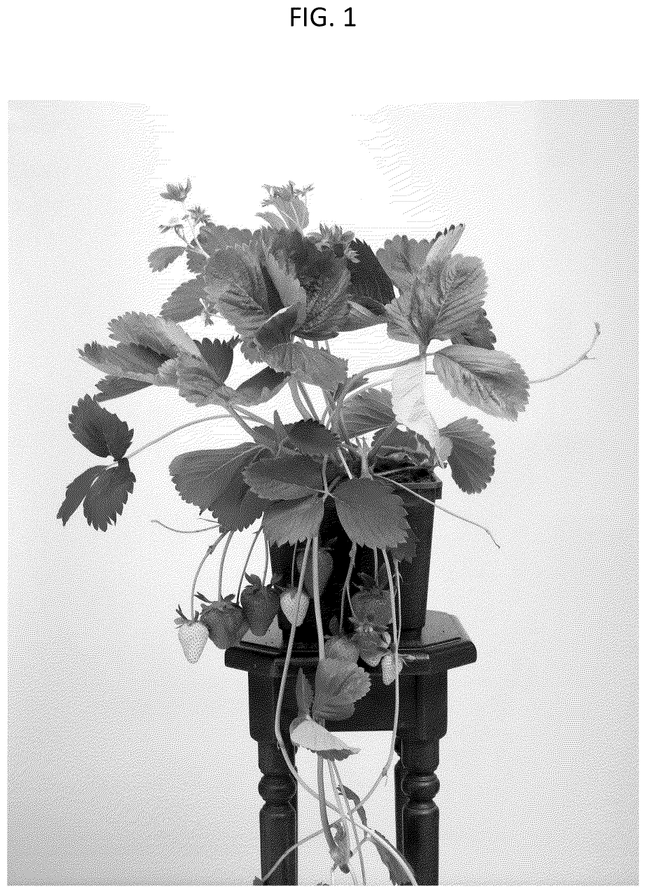

FIG. 2 illustrates, as nearly true as it is reasonably possible to make the same in color photographs of this type, the typical foliage of ‘Florencia’.

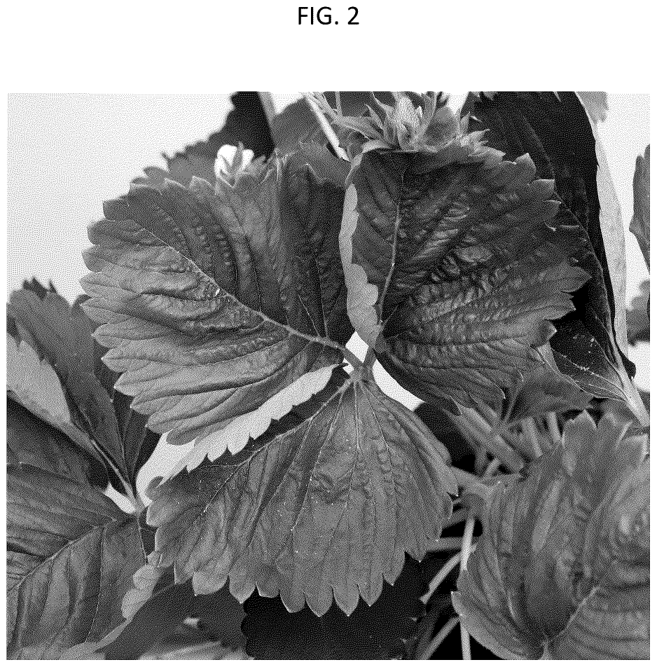

FIG. 3 illustrates, as nearly true as it is reasonably possible to make the same in color photographs of this type, the typical flower of ‘Florencia’.

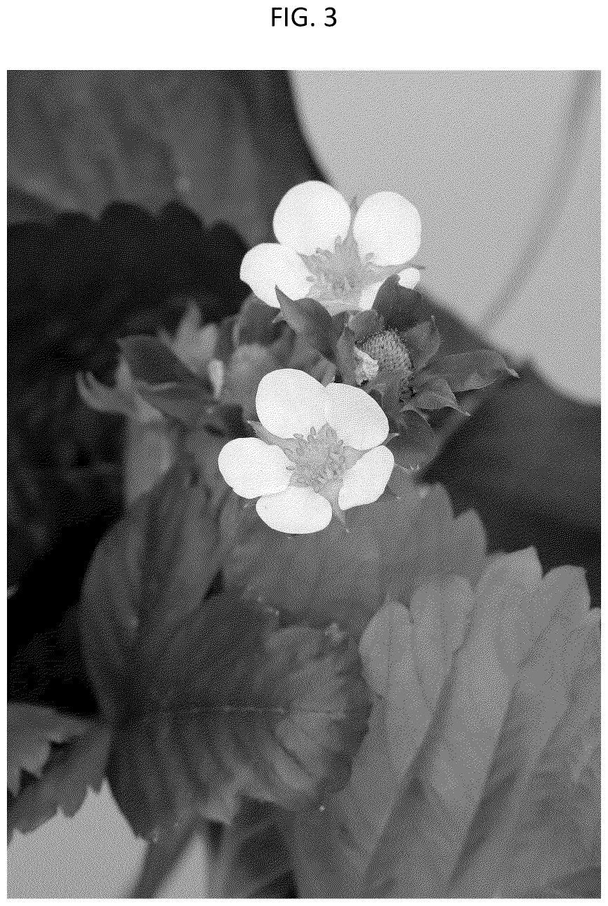

FIG. 4 illustrates, as nearly true as it is reasonably possible to make the same in color photographs of this type, the typical fruit of ‘Florencia’.

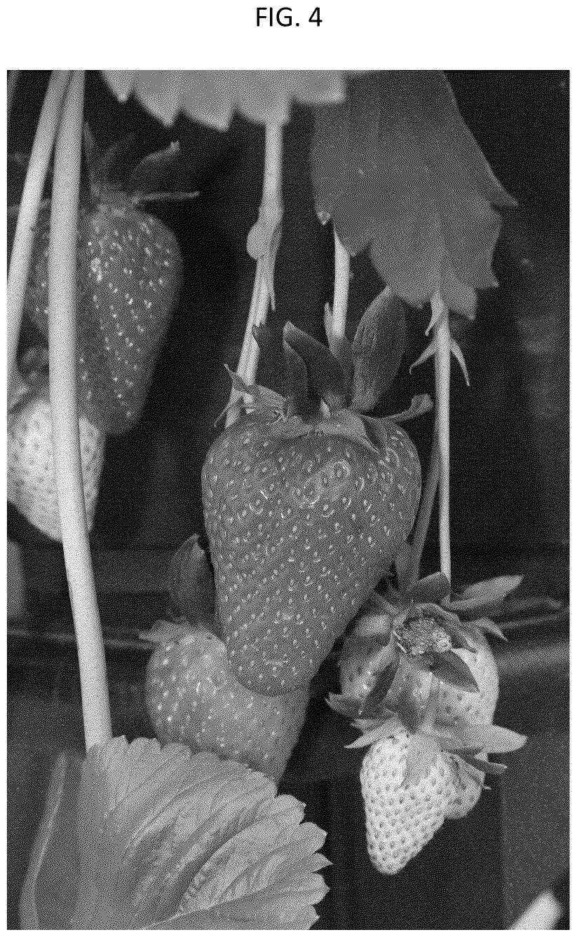

### BOTANICAL DESCRIPTION OF THE PLANT

The following is a detailed botanical description of a new and distinct variety of Fragaria plant known as ‘Florencia’, based upon observations of 12-month-old greenhouse-grown plants in 20 cm nursery containers, produced in Ens, Netherlands. Plants were grown on benches in a non-climate-controlled greenhouse, using conventional production protocols for Fragaria plants which consisted of drip irrigation with fertigation applications as needed. Biological insect control measures were utilized in production. Plants were produced with full sun exposure, and no photoperiodic treatments or artificial light were given to the plants. Temperatures ranged from 7 to 30 degrees Celsius during the day and 7 to 20 degrees at night. Observation data was recorded in May of 2024.

Those skilled in the art will appreciate that certain characteristics will vary with older or, conversely, with younger plants. ‘Florencia’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climatic, and cultural conditions. Color notations are based on The Royal Horticultural Society Colour Chart, The Royal Horticultural Society, London, 2019, reprint.

A botanical description of ‘Florencia’ and a comparison with the parent plants and the most similar commercial Fragaria cultivar known to the inventor, is provided below.
Plant description:
   Growth habit.—Acaulescent, stoloniferous perennial with partially remontant fruiting; with leaves and inflorescence developing from basal rosettes with approximately 3 rosettes per pot; no lateral branching is present.
   Plant profile.—Flattened globular.
   Average height.—31.9 cm to the top of the foliar plane; 33.5 cm to the top of the floral plane.
   Average width.—54.9 cm.
   Propagation.—Mode of propagation — Asexual propagation is accomplished by rooting stolon cuttings.
   Disease resistance.—‘Florencia’ exhibits resistance to the fungal blight which is common in the parent varieties known to be caused by Erysiphaceae.
   Environmental tolerances.—Adapt to temperatures as high as 30 degrees Celsius and prefer soil with high moisture levels.
Root system:
   Type.—Moderately fibrous root system, with adventitious rooting at stolon nodes.
   Branching.—Very freely branched.
   Density.—Moderately dense.
   Distribution.—The majority of the root system is somewhat shallow.
   Diameter.—Largest roots measuring 1.0 mm in diameter.
   Texture.—Smooth; lacking root hairs.
   Color.—White, RHS NN155B.
Stolons:
   Attitude.—Pendulous.
   Cross-section.—Rounded.
   Length.—71.3 cm.
   Diameter.—0.5 cm.
   Internode length.—28.0 cm.
   Texture.—Sparsely covered with pubescence; average length of trichomes is 0.1 cm; colored translucent white, nearest to nearest to RHS NN155D.
   Luster.—Glossy.
   Color.—Juvenile stolons, adaxial and abaxial surfaces — Yellow-green, nearest to RHS 145A and 145B, occasionally tinged greyed-orange on the adaxial surface, nearest to RHS N170B. Mature stolons, adaxial and abaxial surfaces — Yellow-green, nearest to RHS 144A. Internodes, adaxial and abaxial surfaces — Yellow-green, nearest to RHS 144B.
Foliage:
   Arrangement.—Alternate in basal rosettes.
   Attachment.—Petiolate.
   Attitude.—Upward and outward.
   Division.—Trifoliate.
   Compound leaf outline.—Reniform.
   Length.—20.3 cm, excluding the petiole.
   Width.—22.2 cm.
   Quantity.—5 leaves per rosette.
   Leaflets.—Quantity — 3. Shape — Varying from broad ovate to broad obovate. Length — 13.6 cm. Width — 11.9 cm. Cross-section — Concave. Apex — Abruptly acute. Base — Center leaflet is broad acuminate, while the lateral leaflets are unequal broad acuminate to unequal obtuse. Margin — Coarsely dentate to crenate. Texture, adaxial surface — Smooth and glabrous. Texture, abaxial surface — Rugose and glabrous except for the veins which are moderately covered with very thin, soft adpressed pubescence with an average length of 0.3 mm; trichomes are translucent white, nearest to RHS NN155D. Luster, adaxial surface — Glossy. Luster, abaxial surface — Matte. Color — Juvenile foliage color, adaxial surface — Yellow-green, nearest to RHS 144A, transitioning to green, nearest to 143A at the distalmost apices. Juvenile foliage color, abaxial surface — Yellow-green, nearest to RHS 144B; area surrounding venation is yellow-green, 145C. Mature leaf color, adaxial surface — Green, nearest to RHS NN137A. Mature leaf color, abaxial surface — Yellow-green, nearest to RHS 147B. Venation — Type — Pinnate. Vein color, adaxial surface — Yellow-green, nearest to RHS 144B. Vein color, abaxial surface — Yellow-green, nearest to a blend of 145A and 145B.
   Petiole.—Length — 19.6 cm. Diameter — 0.45 cm. Texture, adaxial and abaxial surfaces — Sparsely covered with thin strigose pubescence with an average length of 0.2 cm; trichomes are translucent white, nearest to RHS NN155D. Luster, adaxial and abaxial surfaces — Glossy. Color, adaxial surface — Yellow-green, nearest to a blend of RHS 144A and 144B. Color, abaxial surface — Yellow-green, nearest to a blend of RHS 144A and 144B.
   Stipules.—General — Two present at the base of each leaf. Shape — Lanceolate. Length — Average of 3.0 cm. Width — Average of 0.9 cm. Apex — Abruptly acute. Base — Broad cuneate. Cross-section — Prostrate and convex. Color, adaxial surface — Yellow-green, nearest to RHS 145A transitioning lighter proximal to the petiole, nearest to 145C. Color, abaxial surface — Yellow-green, nearest to RHS 145A transitioning lighter proximal to the petiole, nearest to 145C. Texture, adaxial surface — Glabrous. Texture, abaxial surface — Sparsely covered in pubescence with an average length of 0.1 cm; trichomes are translucent white, nearest to RHS NN155D.
Inflorescence:
   Type.—Compound cymose panicle.
   Natural flowering season.—Late spring through autumn in Ens, Netherlands.
   Time to flower.—9 months.
   Length.—9.8 cm, not including the peduncle.
   Diameter.—8.6 cm.
   Quantity of flowers per inflorescence.—5.
   Quantity of flowers and buds per plant.—20.
   Peduncle.—Length — 24.0 cm. Diameter — 0.4 cm. Arrangement — At an average angle of 40 degrees horizontal. Texture — Sparsely covered with pubescence with an average length of 0.1 cm; trichomes are translucent white, nearest to RHS NN155D. Luster — Glossy. Color, adaxial and abaxial surfaces — Yellow-green, nearest to a blend of RHS 144A and 144B.
Flower buds:
   Shape.—Ovate.
   Length.—Average of 1.4 cm.
   Diameter.—Average of 1.7 cm.
   Texture.—Smooth and glabrous except the immature sepals which are moderately covered with thin, adpressed trichomes with an average length of 0.75 mm; colored translucent white, nearest to RHS NN155D.
   Luster.—Matte.
   Color, adaxial and abaxial surfaces.—Closed bud's sepals appear green, nearest to RHS 143A and petals appear white, NN155A.
Flower:
   Form.—Rotate; single.
   Attitude.—Upright and outward.
   Flower diameter.—3.0 cm.
   Flower depth.—1.0 cm.
   Fragrance.—Moderately strong, sweet fragrance.
   Flowering habit.—Moderately free-flowering.
   Lastingness.—Approximately 7 days.
   Persistent.—Self-cleaning.
   Pedicels.—Length — 3.6 cm. Diameter — 0.175 cm. Cross-section — Carinate. Attitude — Approximately 17.5 degrees to the peduncle axis. Texture — Moderately covered with strigose pubescence with an average length of 0.5 mm; trichomes are translucent white, nearest to RHS NN155D. Luster — Glossy. Color, adaxial and abaxial surfaces — Yellow-green, nearest to RHS 144C.
   Petals.—Arrangement — Rotate with a single whorl of free petals. Quantity of Petals — 5 and occasionally 6. Shape — Broad obovate to near orbicular. Length — 1.3 cm. Width — 1.2 cm. Apex — Obtuse. Base — Cuneate. Margin — Entire; shallowly undulated. Cross-section — Shallowly concave. Texture and luster of upper surface — Glabrous; moderately velvety; matte. Texture and luster of lower surface — Glabrous; moderately velvety; matte. Petal color when opening, upper surface — White, nearest to RHS NN155C, transitioning to NN155A proximally to the base. Petal color when opening, lower surface — White, nearest to RHS NN155C, transitioning to NN155A proximally to the base. Petal color when fully opened, upper surface — White, nearest to RHS NN155D, transitioning to 155B proximally to the base. Petal color when fully opened, lower surface — White, nearest to RHS NN155D, transitioning to 155B proximally to the base. Petal venation color, fully opened, upper surface — Veins are not visible. Petal venation color, fully opened, lower surface — Veins are not visible.
   Calyx.—Shape — Rotate. Length — 0.6 cm. Diameter — 3.0 cm. Sepals — Arrangement — Rotate with sepals in a single whorl and fused at the base. Quantity — 10, on average. Length — 1.4 cm. Width — 0.4 cm. Shape — Elliptic, alternate sepals are ovate. Apex — Acute, alternative sepals are narrow acute. Base — Fused, cuneate. Sepal lobe margin — Entire; not undulated. Texture, upper surface — Sparsely covered with pubescence with an average length of 0.5 mm; trichomes are translucent white, nearest to nearest to RHS N155A. Texture, lower surface — Sparsely covered with pubescence with an average length of 0.625 mm; trichomes are translucent white, nearest to nearest to RHS N155A. Luster, adaxial surface — Matte. Luster, abaxial surface — Matte. Color when opening, upper surface — Green, nearest to RHS 143B, alternative sepals are yellow-green, nearest to 144B. Color when opening, lower surface — Yellow-green, nearest to RHS 144B. Color when fully opened, upper surface — Green, nearest to RHS 143A, alternating sepals are yellow-green, nearest to 144A tipped green, 143A. Color when fully opened, lower surface — Green to yellow-green, nearest to a blend of RHS 143B and 144A.
   Bracts.—Position — One bract present at the base of each pedicel. Type — Single to trifoliate. Shape — Single bracts shaped ovate; compound leaves broad ovate. Cross-section — Carinate. Length — Single bract is 5.6 cm in length and the compound bract is 8.5 cm in length. Width — Single bract is 3.8 cm and the compound bract is 8.9 cm in width. Apex — Acute. Base — Attenuate. Margins — Crenate to dentate. Texture, adaxial surface — Sparsely covered with pubescence with an average length of 0.5 mm; colored translucent white, nearest to nearest to RHS NN155D. Texture, abaxial surface — Moderately covered with pubescence with an average length of 0.75 mm; colored translucent white, nearest to nearest to RHS NN155D. Color, adaxial surface — Green, nearest to a blend of RHS 137C and 143A. Color, abaxial surface — Green, nearest to a blend of RHS 138A and 138B. Bracteoles — Shape — Elliptic to ovate. Apex — Acute. Base — Cuneate. Length — 1.0 cm. Width — 0.5 cm. Cross-section — Shallowly carinate. Texture, adaxial surface — Sparsely covered with pubescence with an average length of 0.5 mm; colored translucent white, nearest to nearest to RHS NN155D. Texture, abaxial surface — Sparsely covered with pubescence with an average length of 0.5 mm; colored translucent white, nearest to nearest to RHS NN155D. Color, adaxial surface — Yellow-green, nearest to RHS 144A. Color, abaxial surface — Yellow-green, nearest to RHS 144B.
Reproductive organs:
   Stamens.—Quantity — Approximately 26. Anthers — Attachment — Basifixed. Shape — Broad elliptic to oblong. Length — 0.15 cm. Width — 0.1 cm. Color — Yellow-green, nearest to RHS 153D. Filaments — Length — Approximately 0.4 cm. Color — Yellow-green, nearest to RHS 150D. Pollen, abundance — Sparse. Pollen, color — Yellow-green, nearest to RHS 153B.
   Pistil.—Quantity — 400. Length — 0.1 cm. Style — Length — 0.8 mm. Color — Green-yellow, nearest to 1B. Stigma — Shape — Club-shaped. Length — 0.2 mm. Diameter — 0.2 mm. Color — Yellow-green, nearest to RHS 151D.
   Ovary color.—Yellow-green, nearest to RHS N144D.
Seed and fruit:
   Fruit.—Harvest Season — Mid-summer through early fall in the Netherlands. Time to maturity — Approximately 5 weeks after flowering. Quantity — An average of 18 per plant. Dimensions — 5.5 cm long and 4.4 cm in diameter. Shape — Long conical. Texture, adaxial and abaxial surfaces — Smooth; glabrous. Luster — Glossy. Color — Immature fruit — Yellow-green, nearest to RHS N144D. Immature flesh — Green-yellow, nearest to RHS 1B. Mature fruit — Red, nearest to RHS 45A. Mature flesh — Red, nearest to RHS 45A. Fruit flavor — A pleasant mixture of slightly sweet and moderately acidic flavors. Firmness of flesh — Firm. Lastingness of fruit — Approximately 1 week. Market use — Fresh fruit market.
   Achenes.—Position — Level or slightly below the fruit surface. Quantity of achenes per fruit — Approximately 300. Shape — Flattened; outline is ovate. Length — 0.1 cm. Diameter — 0.08 cm. Texture — Smooth; glabrous. Color — Yellow-green, nearest to RHS 151A.

### Comparison With the Parents

Plants of the new cultivar ‘Florencia’ may be distinguished from its seed parent, Fragaria x ananassa ‘14-168’ (not patented), by the characteristics described in Table 1.

TABLE 1
Comparison Between ‘Florencia’ and ‘14-168’

| Characteristic | ‘Florencia’ | ‘14-168’ |
|---|---|---|
| Timing of the | Earlier than ‘14-168’. | Later than ‘Florencia’. |
| season's first | | |
| fruit. | | |
| Fruit shape. | Long-conical. | Conical to wedge. |
| General | Red. | Orange-red. |
| coloration of | | |
| the fruit. | | |
| Fruit size. | Larger than the seed | Smaller than |
| | parent. | 'Florencia'. |
| Fruit taste. | A pleasant mixture of | A mixture of moderately |
| | slightly sweet and | sweet and very acidic |
| | moderately acidic | flavors. |
| | flavors. | |

Plants of the new cultivar ‘Florencia’ may be distinguished from its pollen parent, Fragaria x ananassa ‘14-149’ (not patented), by the characteristics described in Table 2.

TABLE 2
Comparison Between ‘Florencia’ and ‘14-149’

| Characteristic | ‘Florencia’ | ‘14-149’ |
|---|---|---|
| Timing of the | Later than ‘14-149’. | Earlier than ‘Florencia’. |
| season'sbfirst | | |
| fruit. | | |
| Fruit shape. | Long-conical. | Wedge. |
| Fruit taste. | A pleasant mixture of | A pleasant sweetness |
| | slightly sweet and | with little to no |
| | moderately acidic | acidic flavor. |
| | flavors. | |

### Comparison With the Closest Known Commercial Comparator

Plants of the new cultivar ‘Florencia’ may be distinguished from the most similar known commercial comparator, Fragaria x ananassa ‘Finette’ (U.S. patent application Ser. No. 19/320,627), by the characteristics described in Table 3.

TABLE 3
Comparison Between ‘Florencia’ and ‘Finette’

| Characteristic | ‘Florencia’ | Finette' |
|---|---|---|
| Timing of the | Earlier than ‘Finette’. | Later than ‘Florencia’. |
| season's first | | |
| fruit. | | |
| Fruit size. | Larger than ‘Finette’. | Smaller than ‘Florencia’. |
| Fruit shape. | Long-conical. | Conical to short-wedge. |
| Fruit taste. | A pleasant mixture of | Sweet with no acidic |
| | slightly sweet and | flavor. |
| | moderately acidic | |
| | flavors. | |

That which is claimed is:

1. A new and distinct cultivar of Fragaria x ananassa plant named ‘Florencia’, substantially as described and illustrated herein.
